(19) United States
(12) Patent Application Publication
(10) Pub. No.: US 2008/0065197 A1
(43) Pub. Date: Mar. 13, 2008

(54) Bifurcated Stent

(75) Inventors: Michael P. Meyer, Richfield, MN (US); Thomas E. Broome, Prior Lake, MN (US); Kevin Grotheim, St. Paul, MN (US); Amnon Yadin, Kfar Vitkin (IL)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 11/853,371

(22) Filed: Sep. 11, 2007

Related U.S. Application Data
(60) Provisional application No. 60/844,011, filed on Sep. 12, 2006.

(51) Int. Cl. A61F 2/82

(52) U.S. Cl. 623/1.16

(57) ABSTRACT

In some embodiment, a stent comprises a plurality of interconnected framework members defining a plurality of cells. A portion of the interconnected framework members comprise plurality of serpentine bands which comprise connected turns and unconnected turns, wherein at least one of the serpentine bands comprises a repeating pattern of three band struts and then five band struts extending between connected turns as the serpentine band is traversed. A portion of the interconnected framework members comprise a side branch structure defining an inner side branch cell that is shaped differently than other cells of the stent. The side branch structure includes a serpentine ring that comprises alternating struts and turns. The turns comprise alternating inner turns and outer turns. The inner turns comprise alternating first inner turns and second inner turns, wherein the second inner turns are located farther away from a side branch center point than the first inner turns.

### CROSS-REFERENCE TO RELATED APPLICATIONS

This application claims the benefit of and priority to U.S. Provisional Application No. 60/844,011, filed Sep. 12, 2006, the entire disclosure of which is hereby incorporated herein by reference in its entirety.

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, for example using “minimally invasive techniques” in which the stent in a radially reduced configuration, optionally restrained in a compressed configuration by a sheath and/or catheter, is delivered by a stent delivery system or introducer to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In at least one embodiment, a stent comprises a plurality of interconnected framework members defining a plurality of cells. A portion of the interconnected framework members comprise a side branch structure defining an inner side branch cell that is shaped differently from other cells of the stent. The side branch structure comprises a serpentine ring that extends around the inner side branch cell. The serpentine ring comprises alternating struts and turns. The turns comprise alternating inner turns and outer turns, and the inner turns comprise alternating first inner turns and second inner turns. The second inner turns are located farther away from a side branch center point than the first inner turns.

In at least one other embodiment, a stent comprises a plurality of interconnected framework members defining a plurality of cells. A portion of the interconnected framework members comprise a side branch structure defining an inner side branch cell that is shaped differently from other cells of the stent. The side branch structure comprises a serpentine ring that extends around the inner side branch cell. The serpentine ring comprises alternating struts and turns. The struts comprise longer struts and shorter struts, and the serpentine ring comprises a repeating pattern of two adjacent longer struts and two adjacent shorter struts.

In at least one other embodiment, a stent comprises a plurality of interconnected framework members defining a plurality of cells. A portion of the interconnected framework members comprise a side branch structure defining an inner side branch cell that is shaped differently from other cells of the stent. The side branch structure comprises a serpentine ring extending around the inner side branch cell and a plurality of connectors. Each connector is connected at one end to the serpentine ring and connected at the other end to another part of the stent. The serpentine ring comprises alternating struts and turns, wherein the first two struts of the serpentine ring located on one side of a connector are parallel to the connector.

In at least one other embodiment, a stent comprises a plurality of interconnected framework members defining a plurality of cells. A portion of the interconnected framework members comprise a side branch structure defining a side branch cell that is shaped differently from other cells of the stent. A portion of the interconnected framework members comprise plurality of serpentine bands and a plurality of connector columns. Each serpentine band comprises a plurality of alternating straight band struts and turns. Adjacent serpentine bands are connected across a connector column by a plurality of connector struts. Each connector strut is connected at one end to a turn of one serpentine band, and is connected at the other end to a turn of another serpentine band. The turns of a serpentine band comprise connected turns that connect to a connector strut and unconnected turns that do not connect to a connector strut. At least one of the serpentine bands comprises a repeating pattern of three band struts and then five band struts extending between connected turns as the serpentine band is traversed.

In at least one other embodiment, a stent comprises a plurality of interconnected framework members defining a plurality of cells. A portion of the interconnected framework members comprise plurality of serpentine bands. Each serpentine band comprises a plurality of alternating straight band struts and turns. Adjacent serpentine bands are connected by at least one connector strut. A portion of the turns comprise connected turns, which connect to a connector strut. At least one of the serpentine bands comprises a repeating pattern of three band struts and then five band struts extending between connected turns as the serpentine band is traversed. A portion of the interconnected framework members comprise a side branch structure defining an inner side branch cell that is shaped differently than other cells of the stent. The side branch structure comprises a serpentine ring extending around the inner side branch cell. The serpentine ring comprises alternating struts and turns. The turns comprise alternating inner turns and outer turns. The inner turns comprise alternating first inner turns and second inner turns, wherein the second inner turns are located farther away from a side branch center point than the first inner turns.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE DRAWINGS

A detailed description of the invention is hereafter described with specific reference being made to the drawings.

FIG. 1 shows a flat pattern for an embodiment of a stent.

FIG. 2 shows the side branch structure of the embodiment of FIG. 1 in greater detail.

FIG. 3 shows a side view of an embodiment of a stent in an unexpanded configuration.

FIG. 4 shows a side view of an embodiment of a stent in an expanded configuration.

FIG. 5 shows an axonometric view of an embodiment of a stent in an expanded configuration.

FIG. 6 shows a top view of an embodiment of a stent in an expanded configuration.

FIG. 7 shows an end view of an embodiment of a stent in an expanded configuration.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

Some examples of stents having a side opening and methods of deploying such stents are disclosed in U.S. Pat. No. 5,596,020 and U.S. Pat. No. 6,835,203, the entire disclosures of which are hereby incorporated herein in their entireties.

The entire disclosures of U.S. Pat. No. 5,922,021, U.S. Pat. No. 6,123,721, U.S. Pat. No. 6,334,870, U.S. Pat. No. 6,478,816, U.S. Pat. No. 6,348,065 and U.S. Pat. No. 6,325,826 are hereby incorporated herein by reference in their entireties. The entire disclosures of U.S. patent application Ser. Nos. 11/604,613, 11/752,837, 11/519,552 and 60/859,460 are hereby incorporated herein by reference in their entireties.

For the purposes of this disclosure, like reference numerals in the figures shall refer to like features unless otherwise indicated.

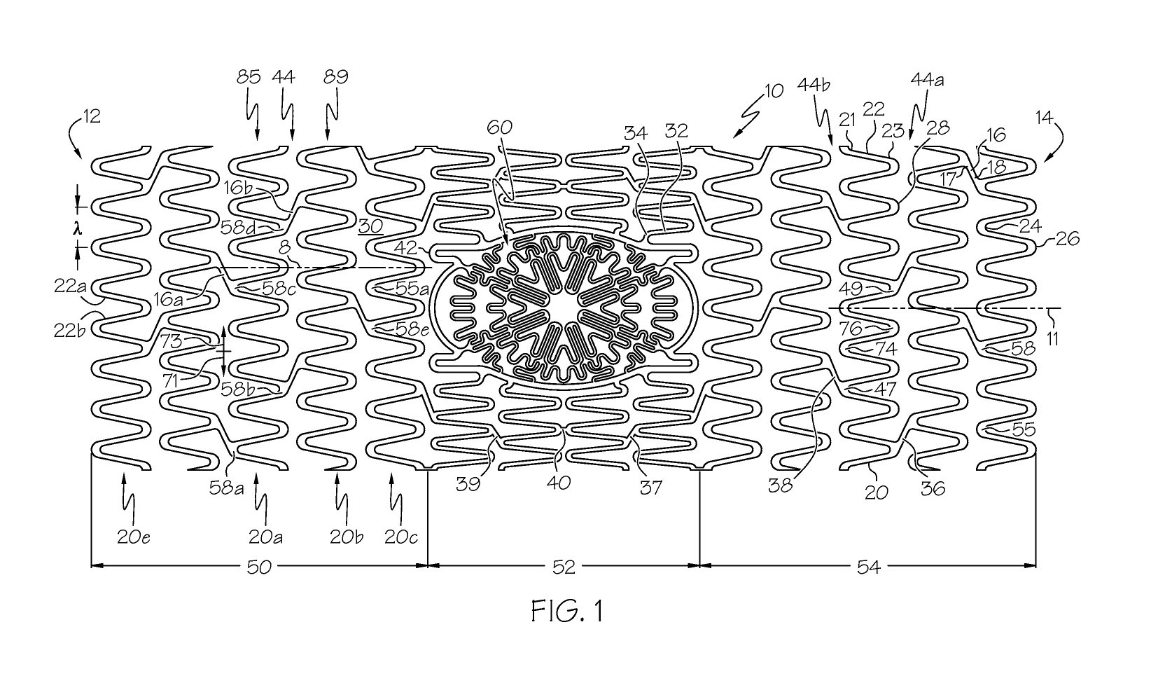

FIG. 1 shows a flat pattern for an embodiment of a stent 10 having a proximal end 12, a distal end 14 and a plurality of serpentine bands 20. Each serpentine band 20 comprises a plurality of struts 22, each strut 22 having a first end 21 and a second end 23. Circumferentially adjacent struts 22 within a serpentine band 20 are connected by turns 28. Turns 28 that point toward the proximal end 12 of the stent 10 comprise proximal peaks 24, and turns 28 that point toward the distal end 14 of the stent 10 comprise distal valleys 26. Each serpentine band 20 extends around at least a portion of a circumference of the stent 10.

A stent 10 can have any suitable number of serpentine bands 20. In various embodiments, a serpentine band 20 can have any suitable number of struts 22 and any suitable number of turns 28. In some embodiments, a serpentine band 20 can have a constant wavelength λ or distance between repeating elements of the serpentine band 20. For example, a wavelength λ may comprise a distance between adjacent proximal peaks 24 of a serpentine band 20, or a distance between adjacent distal valleys 26 of a serpentine band 20. In some embodiments, a stent 10 includes one or more serpentine bands 20 that have a wavelength λ that is different from the wavelength λ of one or more other serpentine bands 20.

A serpentine band 20 can span any suitable distance along the length of the stent 10. In some embodiments, the proximal peaks 24 of a given serpentine band 20 are aligned around a circumference of the stent 10, and the distal valleys 26 are similarly aligned around another circumference of the stent 10. In some embodiments, various peaks 24 can be offset from other peaks 24 within a given serpentine band 20, and various valleys 26 can be offset from other valleys 26 within the band 20.

Each strut 22 comprises a width, which may be measured in a direction normal to the length of the strut 22. In some embodiments, all struts 22 within a given serpentine band 20 have the same width. In some embodiments, the width of various struts 22 within a serpentine band 20 can change. In some embodiments, the width of struts 22 of one serpentine band 20 can be different from the width of struts 22 of another serpentine band 20.

Each turn 28 has a width, which may be measured in a direction normal to the side of the turn 28 (e.g. normal to a tangent line). In some embodiments, the width of a turn 28 can be greater than the width of one or more struts 22 of the stent 10. In some embodiments, the width of a turn 28 can be less than the width of one or more struts 22 of the stent 10. In some embodiments, the width of a turn 28 varies from one end of the turn 28 to the other. For example, a turn 28 can connect to a strut 22 at one end having the same width as the strut 22. The width of the turn 28 increases, and in some embodiments reaches a maximum at a midpoint of the turn 28. The width of the turn 28 then decreases to the width of another strut 22, which may be connected to the second end of the turn 28.

Serpentine bands 20 that are adjacent to one another along the length of the stent 10 are connected by at least one connector strut 16. In some embodiments, a connector strut 16 spans between turns 28 of adjacent serpentine bands 20. For example, a first end 17 of a connector strut 16 can connect to a distal valley 26 of one serpentine band 20, and a second end 18 of the connector strut 16 can connect to a proximal peak 24 of an adjacent serpentine band 20.

Connector struts 16 can connect to any portion of a serpentine band 20, such as a turn 28, or in some embodiments, a strut 22. In some embodiments, a connector strut 16 is linear or straight along its length. In some embodiments, a connector strut 16 can include curvature along its length, and can further include multiple portions of curvature, for example a convex portion and a concave portion that may be connected at an inflection point.

In some embodiments, a connector strut 16 can comprise a first type of connector strut 36 or a second type of connector strut 38. A first connector strut 36 may extend in a first direction. The first connector strut 36 may be oriented at a first angle to a stent lengthwise axis 11. A second connector strut 38 may extend in a second direction that is different than or non-parallel to the first direction. In some embodiments, the first angle and the second angle may have the same magnitude but different orientations. For example, a first connector strut 36 may form a 70° angle with a stent lengthwise axis 11, while a second connector strut 38 may form a negative 70° angle with the stent lengthwise axis 11. In some embodiments, a first angle may comprise a mirror image of a second angle across a line parallel to the stent lengthwise axis 11. In some embodiments, first type of connector strut 36 can have a different shape than second type of connector strut 38.

In some embodiments, a stent 10 further comprises a third type of connector strut 37, a fourth type of connector strut 39 and a fifth type of connector strut 40. Each type of connector strut 36, 37, 38, 39, 40 may be different from each other type of connector strut 36, 37, 38, 39, 40 in at least one way, for example varying in length, width, orientation, etc. As shown in FIG. 1, in some embodiments, third connector struts 37 have an orientation similar to first connector struts 36 but have a shorter length; fourth connector struts 39 have an orientation similar to second connector struts 38 but have a shorter length; and fifth connector struts 40 comprise a connection between two longitudinally aligned turns 28.

A stent 10 further comprises a plurality of cells 30. A cell 30 comprises an opening in the wall portion of the stent 10 oriented between the expandable framework members. In some embodiments, a cell 30 may be bounded by a serpentine band 20, a connector strut 16, another serpentine band 20 and another connector strut 16.

A stent 10 comprises a first end region 50, a central region 52 and a second end region 54. Each region 50, 52, 54 extends across a portion of the length of the stent 10. Each region 50, 52, 54 includes a plurality structural framework elements, for example a plurality of serpentine bands 20. In some embodiments, all of the serpentine bands 20 within a given region 50, 52, 54 are similar in size and shape. In some embodiments, various serpentine bands 20 within a given region 50, 52, 54 may be different in size, shape, strut width, wavelength λ, etc. For example, in some embodiments, serpentine bands 20 located in the central region 52 span a greater distance along the length of the stent 10 than serpentine bands 20 located in the end regions 50, 54. In some embodiments, the struts 22 of serpentine bands 20 located in the central region 52 have a greater length than struts 22 located in the end regions 50, 54. In some embodiments, the struts 22 of serpentine bands 20 located in the end regions 50, 54 are wider than struts 22 located in the central region 52. In some embodiments, the wavelength λ of serpentine bands 20 located in the central region 52 is less than the wavelength λ of serpentine bands 20 located in the end regions 50, 54.

In some embodiments, an area of the stent 10 located between two adjacent serpentine bands 20 can be considered a connector column 44. Each connector column 44 comprises a plurality of connector struts 16. In some embodiments, each connector strut 16 in a connector column 44 can be similar to one another. For example, each connector strut 16 in a first connector column 44a can comprise a first type of connector strut 36. Each connector strut 16 in a second connector column 44b can comprise a second type of connector strut 38.

In some embodiments, first connector columns 44a and second connector columns 44b can alternate along the length of the stent 10. Thus, each interior serpentine band 20 can be positioned between a first connector column 44a and a second connector column 44b. Accordingly, connector struts 16 that connect to one side of a serpentine band 20 can comprise first connector struts 36, and connector struts 16 that connect to the other side of the serpentine band 20 can comprise second connector struts 38.

Turns 28 can comprise connected turns 58 or unconnected turns 55 depending upon whether the turn 28 connects to a connector strut 16. Similarly, proximal peaks 26 can comprise connected proximal peaks 47 or unconnected proximal peaks 74, and distal valleys 26 can comprise connected distal valleys 49 or unconnected distal valleys 76.

A serpentine band 20 can have more unconnected turns 55 than connected turns 58. In some embodiments, a serpentine band 20 has three unconnected turns 55 for each connected turn 58. The 3:1 ratio of unconnected turns 55 to connected turns 58 can also apply to the proximal peaks 24 and to the distal valleys 26.

In some embodiments, as a serpentine band 20 is traversed, there is a repeating pattern of x number of unconnected turns 55 between one connected turn 58 and the next connected turn 58, and then y number of unconnected turns until the next connected turn 58, wherein y is greater than x. For example, referring to FIG. 1, as a serpentine band 20a is traversed from a first connected turn 58a to a second connected turn 58b, there are two unconnected turns 55. Thus, x can equal two. As the serpentine band 20a is traversed from the second connected turn 58b to a third connected turn 58c, there are four unconnected turns 55. Thus, y can equal four. The pattern will then repeat, with x=2 unconnected turns 55 between the third connected turn 58c and a fourth connected turn 58d, etc. In some embodiments, y is a multiple of x, for exampley=2×.

In some embodiments, starting from a connected turn 58, a serpentine band 20 can comprise three band struts 22 between the connected turn 58 and the next connected turn 58 in a first direction. The serpentine band 20 can further comprise five band struts 22 between the connected turn 58 and the next connected turn 58 in a second direction. For example, referring to FIG. 1, a serpentine band 20a includes three band struts 22 between a connected turn 58b and the next connected turn 58a in a first circumferential direction 71. The serpentine band 20a also includes five band struts 22 between the connected turn 58b and the next connected turn 58c in a second circumferential direction 73.

In some embodiments, as a serpentine band 20 is traversed, there can be a repeating pattern of three band struts 22 between one connected turn 58 and the next connected turn 58, and then five band struts 22 until the next connected turn 58. For example, referring to FIG. 1, as a serpentine band 20a is traversed from a first connected turn 58a to a second connected turn 58b, there are three band struts 22. As the serpentine band 20a is traversed from the second connected turn 58b to a third connected turn 58c, there are five band struts 22. The pattern will then repeat, with three band struts 22 between the third connected turn 58c and a fourth connected turn 58d, etc.

In some embodiments, an end serpentine band 20e that is located on the proximal end 13 or the distal end 15 of the stent 10 comprises seven unconnected turns 55 between two connected turns 58. The end serpentine band 20e can further comprise eight band struts 22 between two connected turns 58.

In some embodiments, the connector struts 16 of adjacent connector columns 44 are offset from one another in a stent circumferential direction. For example, one connector strut 16a is offset in a stent circumferential direction from another connector strut 16b located in an adjacent connector column 44. Thus, in some embodiments, a reference line 8 oriented parallel to the stent longitudinal axis 11 that intersects one connector strut 16a will not intersect the other connector strut 16b.

In some embodiments, the band struts 22 of a serpentine band 20 can comprise alternating first band struts 22a and second band struts 22b. In some embodiments, each first band strut 22a is parallel to one another as shown in the flat pattern of FIG. 1. Each second band strut 22b is parallel to one another and non-parallel to the first band struts 22a.

Serpentine bands 20 can comprise a first type of serpentine band 85 and a second type of serpentine band 89. In some embodiments, each first type of serpentine band 85 is aligned with one another such that similar portions of each band 85 align along the length of the stent 10. Each second type of serpentine band 89 is aligned with one another such that similar portions of each band 89 align along the length of the stent 10. Each first type of serpentine band 85 is offset from each second type of serpentine band 89 such that similar portions of the different types of bands 85, 89 are not aligned along the length of the stent.

In some embodiments, the first type of serpentine band 85 and the second type of serpentine band 89 can alternate along at least a portion of the length of the stent 10. Thus, serpentine bands 20 that are located adjacent to one another along the length of the stent 10 can be offset from one another in a stent circumferential direction. Every other serpentine band 20 can be aligned with one another in a stent circumferential direction. For example, a stent 10 can comprise a first serpentine band 20a, a second serpentine band 20b and a third serpentine band 20c along its length. The first and third serpentine bands 20a, 20c comprise a first type of serpentine band 85, and the second serpentine band 20b comprises a second type of serpentine band 89. The first serpentine band 20a is offset from the second serpentine band 20b in a stent circumferential direction. Thus, a reference line 8 extending parallel to the stent longitudinal axis 11 will not intersect similar portions of the first serpentine band 20a and the second serpentine band 20b. As shown, the reference line 8 bisects a distal valley 26 of the first serpentine band 20a but does not bisect a distal valley 26 of the second serpentine band 20b. The second serpentine band 20b is similarly offset from the third serpentine band 20c. The first serpentine band 20a and the third serpentine band 20c are aligned with one another in a stent circumferential direction. Thus, the reference line 8 bisects a distal valley 26 of both the first serpentine band 20a and the third serpentine band 20c.

One serpentine band 20 of a given type 85, 89 can have connected turns 58 that are aligned with unconnected turns 55 of another serpentine band 20 of the same type 85, 89 along the length of the stent 10. For example, the first serpentine band 20a of FIG. 1 includes a connected turn 58c that is longitudinally aligned with an unconnected turn 55a of the third serpentine band 20c.

One serpentine band 20 of a given type 85, 89 can have connected turns 58 that are offset from connected turns 58 of the next adjacent serpentine band 20 of the same type 85, 89 by one proximal peak or one distal valley. For example, the first serpentine band 20a of FIG. 1 includes a connected proximal peak 58c that is offset from a connected proximal peak 58e of the third serpentine band 20c by one proximal peak 24. Thus, in some embodiments, the connector struts 16 of adjacent similar types of connector columns 44a, 44b are offset from one another in the stent circumferential direction by an amount equal to the spacing between adjacent proximal peaks 24 or between adjacent distal valleys 26 in a serpentine band 20.

The central region 52 further comprises a side branch structure 60 and a side branch support ring 42. In various embodiments, some or all of the serpentine bands 20 located in the central region 52 extend about a portion of the stent circumference, while the remainder of the circumference is occupied by the side branch structure 60 and the support ring 42. Thus, some serpentine bands 20 can comprise partial serpentine bands that extend around a portion of the stent circumference.

In some embodiments, partial serpentine bands 20 located in the central region 52 attach directly to a portion of the support ring 42. For example, a first end of a partial serpentine band can connect to one location of the support ring 42, and a second end of the partial serpentine band can connect to another location of the support ring 42.

In some embodiments, a serpentine band 20 comprises one or more shorter struts 32. A shorter strut 32 is generally shorter than other struts 22 of the serpentine band 20. Shorter struts 32 can be located in proximity to the side branch structure 60, and in some embodiments, a shorter strut 32 can connect to a portion of the side branch structure 60. A serpentine band 20 can also comprise one or more offset turns 34, which can connect to one or more shorter struts 32 and can further connect to the support ring 42. An offset turn 34 is generally offset from other turns 28 of the serpentine band 20 that face the same direction (e.g. point toward the same direction). For example, most of the distal valleys 26 of a serpentine band 20 may be aligned about a reference circumference of the stent 10, while an offset distal valley 34 located in the same serpentine band 20 is not aligned on the aforementioned reference circumference.

In various embodiments, serpentine bands 20 located in the central region 52 can comprise any suitable combination of struts 22 and turns 28, including struts of varying length, struts having curvature and turns having any suitable location and orientation.

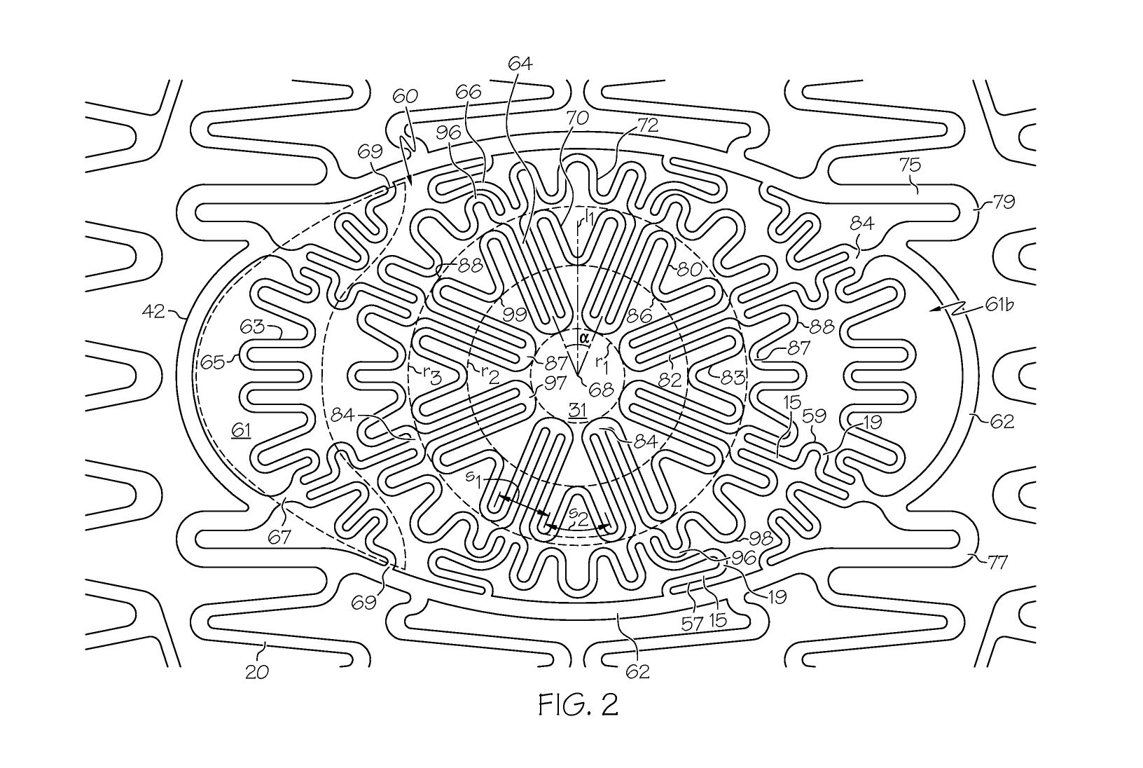

FIG. 2 shows the side branch structure 60 from the embodiment of FIG. 1 in greater detail. The side branch structure 60 comprises a first serpentine ring 70, a second serpentine ring 72, a plurality of side branch inner connectors 64 and a plurality of side branch outer connectors 66. Each serpentine ring 70, 72 comprises a plurality of alternating struts 80 and turns 86. Each inner connector 64 connects between the first serpentine ring 70 and the second serpentine ring 72. The serpentine rings 70, 72 are also referred to herein as side branch rings.

The first serpentine ring 70 extends around and defines an inner side branch cell 31. The inner side branch cell 31 is shaped differently from all other cells 30 of the stent 10. A side branch center point 68 comprises the center of the inner side branch cell 31. The side branch rings 70, 72 are centered upon the side branch center point 68.

The struts 80 of the first serpentine ring 70 comprise first inner struts 82 and second inner struts 83. The first inner struts 82 are longer than the second inner struts 83. Each first inner strut 82 has the same length, and each second inner strut 83 has the same length.

The struts 80 of the first serpentine ring 70 are arranged in a repeating pattern of two adjacent first inner struts 82 and then two adjacent second inner struts 83. The repeating pattern is encountered as the first serpentine ring 70 is traversed around its periphery. Thus, a reference circle that intersects all of the struts 80 of the first serpentine ring 70 would intersect the struts 80 in a repeating pattern of a first inner strut 82, another first inner strut 82, a second inner strut 83, another second inner strut 83, a first inner strut 82, another first inner strut 82, a second inner strut 83, another second inner strut 83, etc.

The turns 86 of the first serpentine ring 70 comprise alternating inner turns 87 and outer turns 88. Thus, in at least one embodiment, the turns 86 located on either side of an inner turn 87 comprise outer turns 88, and the turns 86 located on either side of an outer turn 88 comprise inner turns 87. Turns 86 that point inward with respect to the side branch, for example pointing toward the side branch center point 68, comprise inner turns 87. Turns 86 that point outward with respect to the side branch, for example pointing away from the side branch center point 68, comprise outer turns 88.

The inner turns 87 further comprise alternating first inner turns 97 and second inner turns 99. Thus, in at least one embodiment, the inner turns 87 located on either side of a first inner turn 97 comprise second inner turns 99, and the inner turns 87 located on either side of a second inner turn 99 comprise first inner turns 97. The first inner turns 97 are located closer to the side branch center point 68 than the second inner turns 99. The first inner turns 97 are each located an equal distance away from the side branch center point 68, and thus can be considered aligned around a first reference circle r₁ centered upon the side branch center point 68. The first inner turns 97 are also equally distributed around the circumference of the first reference circle r₁. In some embodiments, the first inner turns 97 span a greater distance than the second inner turns 99, wherein the ends of the first inner turns 97 are farther away from one another than the ends of the second inner turns 99.

The second inner turns 99 are each located an equal distance away from the side branch center point 68, and thus can be considered aligned around a second reference circle r₂ centered upon the side branch center point 68. The second inner turns 99 are equally distributed around the circumference of the second reference circle r₂. The second reference circle r₂ comprises a larger radius than the first reference circle r₁. The first inner turns 97 and the second inner turns 99 are collectively equally spaced around the side branch center point 68. Thus, a reference line 1₁ oriented in a side branch radial direction that bisects a second inner turn 99 will bisect the angle α between two first inner turns 97. Similarly, a line that bisects a first inner turn 97 will bisect an angle formed between the two second inner turns 99 located on either side of the first inner turn 97.

Each first inner strut 82 is connected at an inner end to a first inner turn 97 and is connected at an outer end to an outer turn 88. Each second inner strut 83 is connected at an inner end to a second inner turn 99 and is connected at an outer end to an outer turn 88.

The outer turns 88 are each located an equal distance away from the side branch center point 68, and thus can be considered aligned around a third reference circle r₃ centered upon the side branch center point 68. Adjacent outer turns 88 are spaced around the third reference circle r₃ at alternating first spacing s₁ and second spacing s₂ intervals. Each outer turn 88 is adjacent to two other outer turns 88, one located on a first side (e.g. clockwise around the third reference circle r₃) and the other located on the other side (e.g. counter clockwise around the third reference circle r₃). The outer turn 88 will be separated from one adjacent outer turn 88 by the first spacing s₁ and will be separated from the other adjacent outer turn 88 by the second spacing s₂. Adjacent outer turns 88 that are located on opposite sides of a first inner turn 97 are separated by the first spacing s₁. Adjacent outer turns 88 that are located on opposite sides of a second inner turn 99 are separated by the second spacing s₂. In some embodiments, the second spacing s₂ is greater than the first spacing s₁.

In some embodiments, an inner connector 64 is straight along its length and is oriented in a side branch radial direction. In some embodiments, one inner connector 64 and another inner connector 64 that is located across the inner side branch cell 31 are both oriented upon a common reference line that passes through the side branch center point 68. In some embodiments, all of the inner connectors 64 are evenly distributed around the side branch center point 68.

In some embodiments, each strut 80 of the first side branch ring 70 is parallel to at least one inner connector 64. In some embodiments, an inner connector 64 connects to a first inner turn 97, and the struts 80 that also connect to the first inner turn 97 are parallel to the inner connector 64. In some embodiments, the first two struts 80 located adjacent to an inner connector 64 in either direction are parallel to the inner connector 64. Thus, the first side branch ring 70 can comprise four adjacent struts 80 that are all parallel to an inner connector 64, wherein some of the struts 80 and the inner connector 64 connect to a common turn 86. The four adjacent struts 80 can comprise two first inner struts 82 and two second inner struts 83. The two first inner struts 82 can comprise mirror images of one another taken across the inner connector 64. The two second inner struts 83 can also comprise mirror images of one another taken across the inner connector 64.

The second side branch ring 72 extends around the first side branch ring 70. In some embodiments, the second side branch ring 72 comprises more struts 80 and more turns 86 than the first side branch ring 70. The turns 86 of the second side branch ring 72 comprise alternating inner turns 87 and outer turns 88, wherein the inner turns 87 are located closer to the side branch center point 68 than the outer turns 88. The outer turns 88 can further comprise first outer turns 96 and second outer turns 98, wherein the first outer turns 96 are located closer to the side branch center point 68 than the second outer turns 98. Various embodiments of turns 86 in the second side branch ring 72 can span differing distances, can comprise different arc lengths and can have different radii of curvature.

In some embodiments, an inner connector 64 is connected at an inner end to a turn 86 of the first side branch ring 70 and is connected at an outer end to a turn 86 of the second side branch ring 72. In some embodiments, an inner connector 64 spans between a first inner turn 97 of the first side branch ring 70 and an inner turn 87 of the second side branch ring 72.

An inner turn 87 of the second side branch ring 72 that connects to an inner connector 64 can be different from other inner turns 87 of the second side branch ring 72. For example, an inner turn 87 that connects to an inner connector 64 can span a greater distance than other inner turns 87. An inner turn 87 of the second side branch ring 72 that connects to an inner connector 64 can span the same distance and have the same curvature as a first inner turn 97 of the first side branch ring 70.

In some embodiments, the side branch structure 60 further comprises a junction area 84 where a side branch connector 64, 66 connects to a turn 86. The junction area 84 comprises additional stent material connected to the turn 86 and/or to the side branch connector 64, 66 that provides a greater scaffolding than would be provided by a turn 86 of constant width connected to a side branch connector 64, 66 of constant width.

The stent 10 further comprises ancillary side branch structure 61 and a side branch support ring 42. In some embodiments, the ancillary side branch structure 61 and the side branch support ring 42 can be considered a part of the side branch structure 60.

The ancillary side branch structure 61 comprises additional stent structure adjacent to the side branch rings 70, 72 that is located within the support ring 42. The ancillary side branch structure 61 can have any suitable configuration of stent elements, and in some embodiments comprises a plurality of ancillary struts 63 and a plurality of ancillary turns 65. Various embodiments of ancillary turns 65 can span differing distances, can comprise different arc lengths and can have different radii of curvature. A plurality of the ancillary struts 63 can be oriented parallel to a longitudinal axis 11. A plurality of the ancillary struts 63 can be oriented parallel to an inner connector 64 or at least a portion of an outer connector 66. The ancillary side branch structure 61 can further comprise at least one ancillary connector 67 that connects between an ancillary turn 65 and the support ring 42. The ancillary side branch structure 61 can also comprise at least one junction area 84.

In some embodiments, circumferentially opposed outer ends 69 of an ancillary side branch structure 61 can connect to the support ring 42. Circumferentially opposed “upper” and “lower” halves, as depicted in FIG. 2, of the ancillary side branch structure 61 can comprise mirror images of one another taken across a stent longitudinal axis that passes through the side branch center point 68.

In some embodiments, a first ancillary side branch structure 61 and a second ancillary side branch structure 61b can be located on opposite sides of the side branch rings 70, 72. In some embodiments, the second ancillary side branch structure 61b comprises a mirror image of the first ancillary side branch structure 61 taken across an axis oriented in the stent circumferential direction that passes through the side branch center point 68. In some other embodiments, the first ancillary side branch structure 61 and the second ancillary side branch structure 61b can comprise different structural configurations.

The side branch outer connectors 66 connect between the second side branch ring 72 and another portion of the stent 10. Each side branch outer connectors 66 can comprise a plurality of straight portions 15 and a plurality of turns 19. In some embodiments, a straight portion 15 is parallel to an inner side branch connector 64. In some embodiments, an outer connector 66 connects to a turn 86 of the second side branch ring 72 that also connects to an inner connector 64.

In some embodiments, the side branch outer connectors 66 comprise first outer connectors 57 and second outer connectors 59. The first outer connectors 57 have a different shape than the second outer connectors 59. The first outer connectors 57 connect to the support ring 42. The second outer connectors 59 connect to a portion of the ancillary side branch structure 61, such as an ancillary turn 65, for example an ancillary turn 65 that also connects to an ancillary connector 67.

In some embodiments, one first outer connector 57 can comprise a mirror image of another first outer connector 57 taken across a stent longitudinal axis that intersects the side branch center point 68. A first outer connector 57 can also comprise a mirror image of another first outer connector 57 taken across an axis oriented in the stent circumferential direction that passes through the side branch center point 68. Similarly, one second outer connector 59 can comprise a mirror image of another second outer connector 59 taken across a stent longitudinal axis that intersects the side branch center point 68. A second outer connector 59 can also comprise a mirror image of another second outer connector 59 taken across an axis oriented in the stent circumferential direction that passes through the side branch center point 68.

In some embodiments, outer turns 88 of the second side branch ring 72 that are aligned with a first outer connector 57 in a side branch radial direction comprise first outer turns 96.

The side branch support ring 42 provides a more rigid support to the side branch structure 60 than would otherwise be provided by the serpentine bands 20 alone. In some embodiments, the support ring 42 extends continuously around the side branch structure 60 and side branch outer connectors 66. In some embodiments, the support ring 42 comprises a substantially constant strut width, and in some embodiments, struts of the support ring 42 have a greater width than elements of the serpentine bands 20 or other side branch structure 60. In some embodiments, the support ring 42 comprises a plurality of outer support struts 62. In some embodiments, an outer support strut 62 is curved along its length and is concave with respect to the side branch center point 68.

The support ring 42 further comprises a plurality of loop portions 77. Each loop portion 77 comprises a loop turn 79 and a plurality of loop struts 75. In some embodiments, a loop strut 75 is straight and is oriented parallel to the stent longitudinal axis. In some embodiments, a loop turn 79 is oriented with a peak (e.g. a maximum or minimum) pointed in a stent longitudinal direction. This configuration of loop portions 77 allows the support ring 42 to expand in the stent circumferential direction with lessened longitudinal shortening of the support ring 42 than if the support ring 42 did not include loop portions 77. This configuration can also help to provide apposition between the support ring 42 and areas of a vessel bifurcation, such as an elliptical intersection ring between a primary vessel and a branch vessel, while the support ring 42 remains within the primary vessel.

In some embodiments, one loop portion 77 can comprise a mirror image of another loop portion 77 taken across a stent longitudinal axis that intersects the side branch center point 68. A loop portion 77 can also comprise a mirror image of another loop portion 77 taken across an axis oriented in the stent circumferential direction that passes through the side branch center point 68.

In some embodiments, the stent 10 can comprise other configurations of serpentine bands 20, support ring 42 and/or ancillary side branch structure 61, for example as disclosed in U.S. patent application Ser. Nos. 11/604,613, 11/752,837, 11/848,171 and 11/519,552.

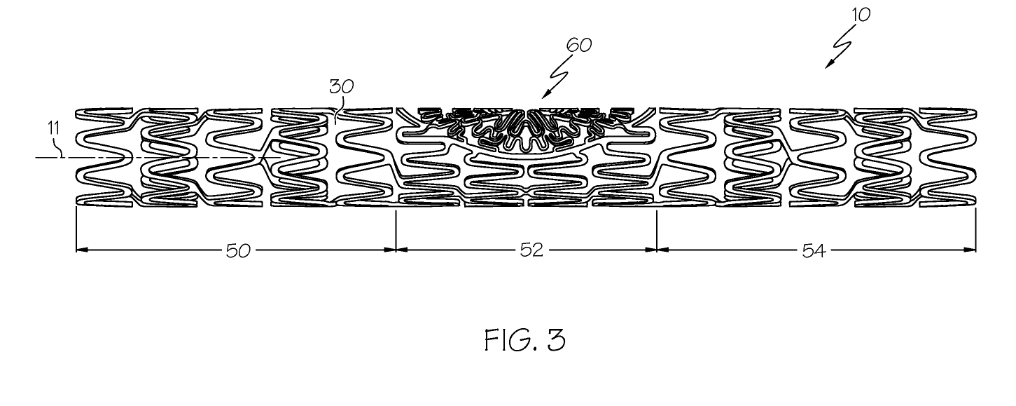

FIG. 3 shows a side view of an embodiment of a stent 10 in an unexpanded configuration, wherein the stent framework forms a substantially cylindrical wall portion having a plurality of cells 30. The side branch structure 60 forms a portion of the substantially cylindrical shape.

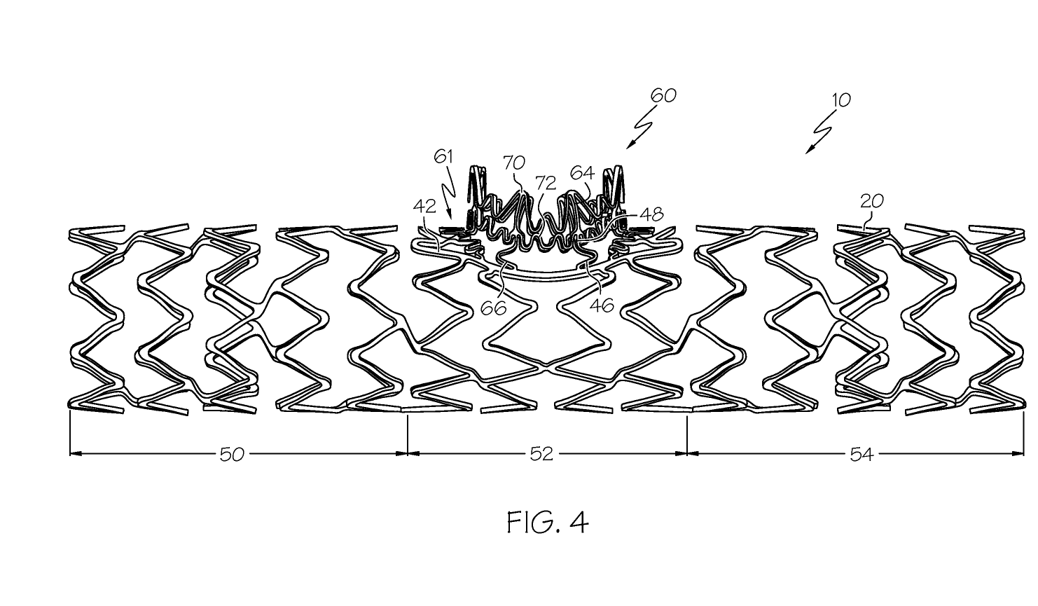

FIG. 4 shows a side view of an embodiment of a stent 10 in an expanded configuration with the side branch structure 60 outwardly deployed. Upon expansion, the serpentine bands 20 radially expand, thereby increasing the diameter of the cylindrical framework formed by the stent 10.

The support ring 42 can also experience expansion in the stent circumferential direction when the stent 10 is expanded. However, in some embodiments, the support ring 42 experiences less expansion in the stent circumferential direction than the serpentine bands 20. For example, in an unexpanded state as shown in FIG. 3, the stent 10 can comprise an unexpanded circumference as measured around an outer perimeter of the stent 10 orthogonal to the stent longitudinal axis 11. The circumferential span of the support ring 42 can account for more than one-half of the unexpanded circumference. Accordingly, the circumferential span of the serpentine bands 20 that comprise the balance of the unexpanded circumference can comprise less than one-half of the unexpanded circumference. After expansion of the stent 10, for example prior to the outward deployment of the side branch structure 60, the stent 10 can comprise an expanded circumference. The circumferential span of the support ring 42 can account for less than one-half of the expanded circumference, while the circumferential span of the serpentine bands 20 that comprise the balance of the expanded circumference is greater than one-half of the expanded circumference.

In some embodiments, the serpentine bands 20 that connect to the support ring 42 can experience more circumferential expansion than the serpentine bands 20 that do not connect to the support ring 42. Thus, upon expansion of the stent 10, the support ring 42 can experience less circumferential expansion than the serpentine bands 20 that do not connect to the support ring 42. A specific amount of circumferential expansion of a stent element 20, 42 can be measured, for example, by defining a unit of circumferential length between two arbitrary points on the stent element 20, 42 prior to expansion. The circumferential distance between the two points after expansion can be compared to the original unit and a percentage or expansion ratio can be calculated for that portion of the stent element 20, 42. The expansion ratio of the support ring 42 can be less than the expansion ratio of the serpentine bands 20. The expansion ratio of a serpentine band 20 located in the central region 52 can be greater than the expansion ratio of a serpentine band 20 located in an end region 50, 54.

Referring again to FIG. 4, the side branch structure 60 is shown outwardly deployed and extending beyond other stent framework in a stent radial direction. Upon outward deployment of the side branch structure 60, the side branch rings 70, 72 displace outwardly. The first side branch ring 70 can experience more outward displacement than the second side branch ring 72. The first side branch ring 70 can also experience an equal or greater increase in size (e.g. diameter) than the second side branch ring 72.

Upon outward deployment of the side branch structure 60, the side branch outer connectors 66 reorient such that a first portion 46 of each outer connector 66 remains a part of the substantially cylindrical stent framework and a second portion of each outer connector 66 extends outwardly from the substantially cylindrical portion of the stent 10 and attaches to the deployed side branch structure 60.

When the stent 10 is oriented within a vessel bifurcation in an expanded configuration with the main cylindrical portion of the stent 10 arranged to support a primary vessel, the deployed side branch structure 60 can extend into and support a side branch vessel. In some embodiments, the side branch structure 60 can be considered the portion of the stent 10 that deploys outwardly from the traditional substantially cylindrical stent framework and becomes arranged to support the side branch vessel. Thus, in some embodiments, the side branch rings 70, 72, the side branch inner connectors 64 and at least the second portion 48 of the side branch outer connectors 66 can comprise the side branch structure 60. Accordingly, the support ring 42 and the ancillary side branch structure 61, which can remain oriented in the substantially cylindrical stent framework, can be considered as excluded from the side branch structure 60.

In some embodiments, the side branch structure 60 need not be defined by an outward deployment beyond the substantially cylindrical stent framework. Thus, in some embodiments, the support ring 42 and the ancillary side branch structure 61 can be considered a part of the side branch structure 60 even when they remain oriented in the substantially cylindrical stent framework. Further, in some embodiments, the side branch structure 60 can be expanded beyond the orientation depicted in FIG. 4. In some embodiments, even the ancillary side branch structure 61 can be outwardly deployed and extend outwardly from the substantially cylindrical stent framework in a stent radial direction.

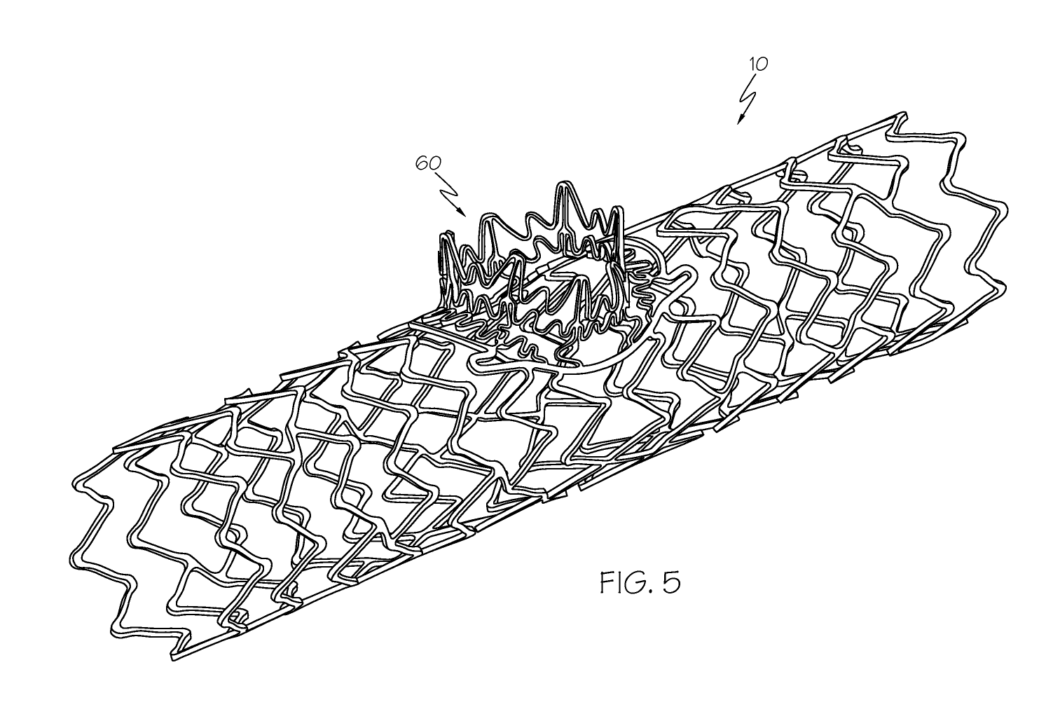

FIG. 5 shows a three-dimensional off-axis view of an embodiment of a stent 10 in an expanded configuration with the side branch structure 60 outwardly deployed.

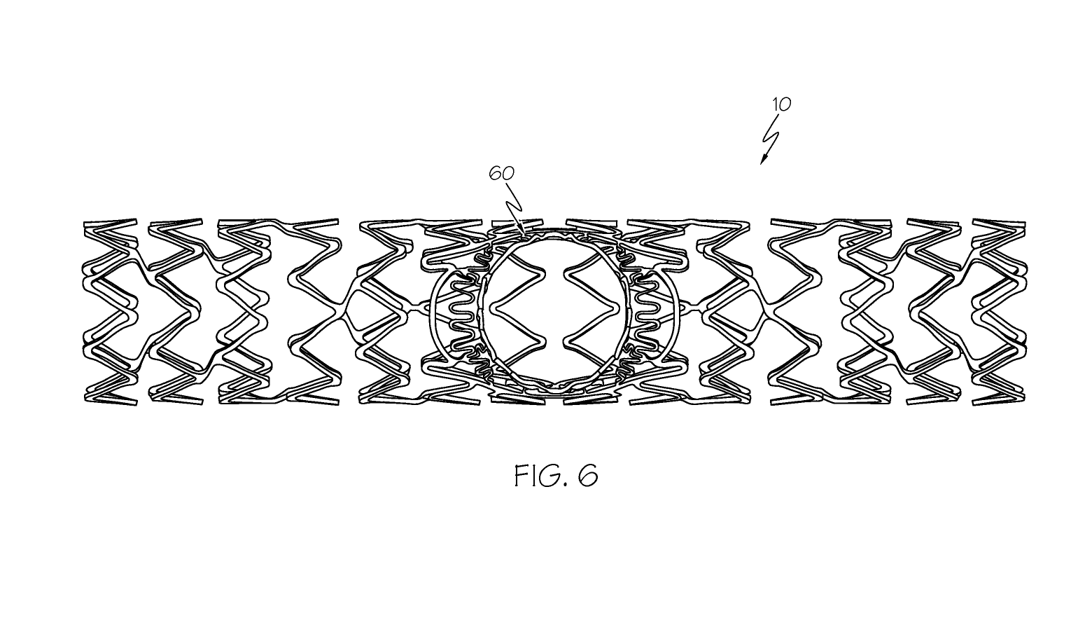

FIG. 6 shows a top view of an embodiment of a stent 10 in an expanded configuration with the side branch structure 60 outwardly deployed.

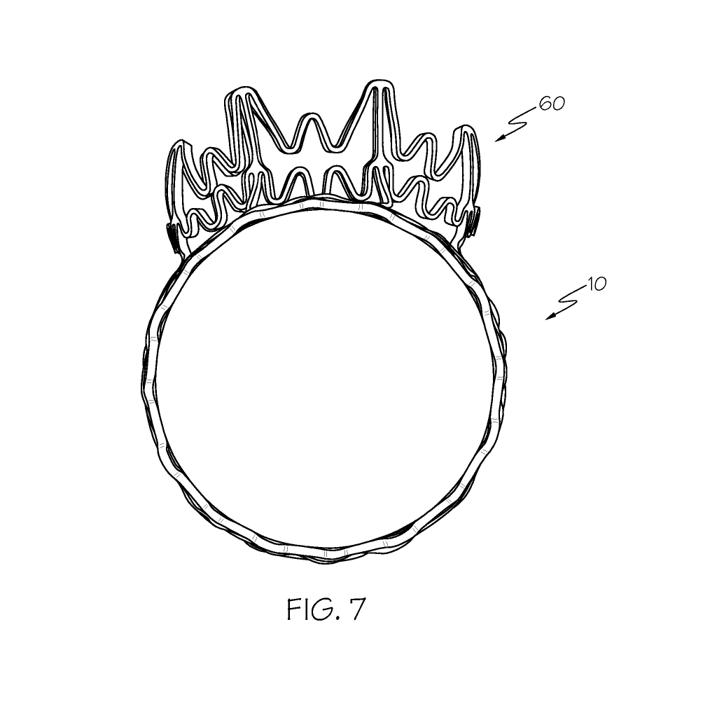

FIG. 7 shows an end view of an embodiment of a stent 10 in an expanded configuration with the side branch structure 60 outwardly deployed. It can be seen that the distance across the deployed side branch structure 60 is slightly less than the distance across the expanded substantially cylindrical portion of the stent 10. In various embodiments, the distance across a deployed side branch structure 60 could be less than or greater than depicted in FIG. 7. In some embodiments, the distance across the deployed side branch structure 60 can even be greater than the distance across the expanded substantially cylindrical portion of the stent 10.

The invention further comprises methods of delivering and expanding stents 10 according to the embodiments disclosed herein.

In some embodiments the stent 10, the delivery system or other portion of the assembly can include one or more areas, bands, coatings, members, etc. that is (are) detectable by imaging modalities such as X-Ray, MRI, ultrasound, etc. In some embodiments at least a portion of the stent 10 and/or adjacent assembly is at least partially radiopaque.

In some embodiments, at least a portion of the stent 10 is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Some other examples of therapeutic agents include everolimus and sirolimus, their analogs and conjugates. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The following numbered paragraphs describe further embodiments of a stent:

1. A stent comprising:

a plurality of interconnected framework members defining a plurality of cells, a portion of the interconnected framework members comprising a side branch structure defining an inner side branch cell, the inner side branch cell being shaped differently than other cells of the stent;
the side branch structure comprising a serpentine ring extending around the inner side branch cell, the serpentine ring comprising alternating struts and turns, the turns comprising alternating inner turns and outer turns, the inner turns comprising alternating first inner turns and second inner turns;
wherein the second inner turns are located farther away from a side branch center point than the first inner turns.
   2. The stent of numbered paragraph 1, wherein the first inner turns are equally distributed around a first reference circle centered upon the side branch center point.
   3. The stent of numbered paragraph 2, wherein the second inner turns are equally distributed around a second reference circle centered upon the side branch center point.
   4. The stent of numbered paragraph 1, wherein the first inner turns and the second inner turns are collectively equally distributed around the side branch center point.
   5. The stent of numbered paragraph 1, wherein an arc length of a first inner turn is equal to or greater than an arc length of a second inner turn.
   6. The stent of numbered paragraph 1, the struts comprising first struts and second struts, the first struts being longer than the second struts.
   7. The stent of numbered paragraph 6, wherein the struts of the serpentine ring form a repeating pattern of two adjacent first struts and two adjacent second struts.
   8. The stent of numbered paragraph 1, further comprising a support ring that extends continuously around the side branch structure.
   9. The stent of numbered paragraph 8, wherein each portion of the support ring comprises a strut width that is greater than the width of struts of the side branch structure.
   10. The stent of numbered paragraph 8, the support ring comprising a plurality of struts oriented in a stent longitudinal direction.
   11. The stent of numbered paragraph 1, wherein said serpentine ring comprises an inner serpentine ring, the side branch structure further comprising an outer serpentine ring, the outer serpentine ring comprising alternating struts and turns, the outer serpentine ring having more struts and more turns than the inner serpentine ring.
   12. The stent of numbered paragraph 11, the side branch structure further comprising a plurality of connectors, each connector connecting between a first inner turn of the inner serpentine ring and an inner turn of the outer serpentine ring.
   13. The stent of numbered paragraph 12, wherein each connector is oriented in a side branch radial direction.
   14. The stent of numbered paragraph 12, wherein the struts of the inner serpentine ring that are located on either side of a connector are parallel to the connector.
   15. A stent comprising:
a plurality of interconnected framework members defining a plurality of cells, a portion of the interconnected framework members comprising a side branch structure defining an inner side branch cell, the inner side branch cell being shaped differently than other cells of the stent;
the side branch structure comprising a serpentine ring extending around the inner side branch cell, the serpentine ring comprising alternating struts and turns, the struts comprising longer struts and shorter struts, the serpentine ring comprising a repeating pattern of two adjacent longer struts and two adjacent shorter struts.
   16. The stent of numbered paragraph 15, the turns of the serpentine ring comprising alternating inner turns and outer turns, the inner turns comprising alternating first inner turns and second inner turns, the second inner turns located farther away from a side branch center point than the first inner turns.
   17. The stent of numbered paragraph 16, wherein the first inner turns connect between two longer struts.
   18. The stent of numbered paragraph 16, wherein the second inner turns connect between two shorter struts.
   19. The stent of numbered paragraph 15, wherein adjacent longer struts are parallel to one another.
   20. A stent comprising:
a plurality of interconnected framework members defining a plurality of cells, a portion of the interconnected framework members comprising a side branch structure defining an inner side branch cell, the inner side branch cell being shaped differently than other cells of the stent;
the side branch structure comprising a serpentine ring extending around the inner side branch cell and a plurality of connectors, each connector connected at one end to the serpentine ring and connected at the other end to another part of the stent, the serpentine ring comprising alternating struts and turns;
wherein the first two struts of the serpentine ring located on one side of a connector are parallel to the connector.

The above disclosure is intended to be illustrative and not exhaustive. This description will suggest many variations and alternatives to one of ordinary skill in this field of art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A stent comprising:
a plurality of interconnected framework members defining a plurality of cells;
a portion of the interconnected framework members comprising a side branch structure defining a side branch cell, the side branch cell being shaped differently than other cells of the stent;
a portion of the interconnected framework members comprising plurality of serpentine bands and a plurality of connector columns, each serpentine band comprising a plurality of alternating straight band struts and turns, adjacent serpentine bands connected across a connector column by a plurality of connector struts, each connector strut connected at one end to a turn of one serpentine band and connected at the other end to a turn of another serpentine band, the turns of a serpentine band comprising connected turns that connect to a connector strut and unconnected turns that do not connect to a connector strut, at least one of the serpentine bands comprising a repeating pattern of three band struts and then five band struts extending between connected turns as the serpentine band is traversed.

2. The stent of claim 1, wherein a plurality of the serpentine bands comprise a repeating pattern of three band struts and then five band struts extending between connected turns.

3. The stent of claim 1, wherein the serpentine bands comprise first serpentine bands and second serpentine bands, turns of the first serpentine bands aligned with one another in a stent longitudinal direction, turns of the first serpentine bands offset from turns of the second serpentine bands in a stent longitudinal direction.

4. The stent of claim 1, wherein the connector columns comprise first connector columns and second connector columns, connector struts of the first connector columns being parallel to one another, connector struts of the second connector columns being nonparallel to the connector struts of the first connector columns.

5. The stent of claim 4, wherein first connector columns and second connector columns alternate between adjacent serpentine bands.

6. The stent of claim 5, wherein an angle between a connector strut of a first connector column and a stent longitudinal axis is equal in magnitude to an angle between a connector strut of a second connector column and the stent longitudinal axis.

7. The stent of claim 1, wherein a portion of the interconnected framework members comprise a support ring that extends continuously around the side branch structure.

8. The stent of claim 7, wherein a portion of the interconnected framework members comprise a plurality of partial serpentine bands, each partial serpentine band connected at one end to the support ring at a first location and connected at the other end to the support ring at a second location.

9. The stent of claim 7, wherein the support ring comprises a plurality of portions that are parallel to a stent longitudinal axis and a plurality of portions that are concave with respect to a center point of the side branch cell.

10. A stent comprising:
a plurality of interconnected framework members defining a plurality of cells;
a portion of the interconnected framework members comprising plurality of serpentine bands, each serpentine band comprising a plurality of alternating straight band struts and turns, adjacent serpentine bands connected by at least one connector strut, a portion of said turns comprising connected turns that connect to a connector strut, at least one of the serpentine bands comprising a repeating pattern of three band struts and then five band struts extending between connected turns as the serpentine band is traversed;
a portion of the interconnected framework members comprising a side branch structure defining an inner side branch cell, the inner side branch cell being shaped differently than other cells of the stent;
the side branch structure comprising a serpentine ring extending around the inner side branch cell, the serpentine ring comprising alternating struts and turns, the turns comprising alternating inner turns and outer turns, the inner turns comprising alternating first inner turns and second inner turns;
wherein the second inner turns are located farther away from a side branch center point than the first inner turns.

11. The stent of claim 10, wherein the first inner turns are equally distributed around a first reference circle centered upon the side branch center point.

12. The stent of claim 11, wherein the second inner turns are equally distributed around a second reference circle centered upon the side branch center point.

13. The stent of claim 10, the struts comprising first struts and second struts, the first struts being longer than the second struts.

14. The stent of claim 13, wherein the struts of the serpentine ring form a repeating pattern of two adjacent first struts and two adjacent second struts.

15. The stent of claim 10, further comprising a support ring that extends continuously around the side branch structure, the support ring comprising a strut width that is greater than the width of struts of the side branch structure.

16. The stent of claim 15, the support ring comprising a plurality of struts oriented in a stent longitudinal direction.

17. The stent of claim 10, wherein said serpentine ring comprises an inner serpentine ring, the side branch structure further comprising an outer serpentine ring, the outer serpentine ring comprising alternating struts and turns, the outer serpentine ring having more struts and more turns than the inner serpentine ring.

18. The stent of claim 17, the side branch structure further comprising a plurality of side branch connectors, each side branch connector connecting between a first inner turn of the inner serpentine ring and an inner turn of the outer serpentine ring.

19. The stent of claim 18, wherein each side branch connector is oriented in a side branch radial direction.

20. The stent of claim 18, wherein the struts of the inner serpentine ring that are located on either side of a connector are parallel to the connector.
